(19) United States
(12) Patent Application Publication
(10) Pub. No.: US 2021/0356464 A1
(43) Pub. Date: Nov. 18, 2021

(54) SEROLOGICAL ASSAYS FOR DIAGNOSING OR CONFIRMING COVID-19 VIRUS INFECTIONS

(71) Applicant: Cellex, Inc., Research Triangle Park, NC (US)

(72) Inventor: Xingxiang LI, Morrisville, NC (US)

(73) Assignee: Cellex, Inc., Research Triangle Park, NC (US)

(21) Appl. No.: 17/308,494

(22) Filed: May 5, 2021

Related U.S. Application Data
(60) Provisional application No. 63/020,321, filed on May 5, 2020.
(60) Provisional application No. 63/066,990, filed on Aug. 18, 2020.

(51) Int. Cl. G01N 33/569; G01N 33/53; G01N 33/543

(52) U.S. Cl. CPC G01N 33/56983; G01N 2333/165; G01N 33/54388; G01N 33/5302

(57) ABSTRACT

Inventions herein described relate to methods, devices and kits for detecting viral infections, particularly COVID-19 infections by the SARs-CoV2 corona virus. Various aspects of the inventions relate to serological antibody tests, particularly those that reduce false positive. Additionally, various aspects relate to serological detection of acute phase immune responses and distinguishing them from later phase response. Additional aspects relate to detection of antibodies indicative of natural COVID-19 virus infection even if the sample is from a vaccinated individual.

### REFERENCE TO RELATED APPLICATIONS

This application claims priority of U.S. Provisional Patent Application No. 63/020,321 filed 5 May 2020 and U.S. Provisional Patent Application No. 63/066,990 filed 18 Aug. 2020 both of which are herein incorporated in their entirety.

### STATEMENT OF GOVERNMENT RIGHTS

No government funds were used in making the invention herein described.

### FIELD OF THE INVENTION

The inventions herein described relate to serological tests for viral infections, particularly infection by SARS CoV-2.

### BACKGROUND

A challenging aspect of developing an COVID-19 specific antibody assay is that most adults have antibodies against non COVID-19 coronaviruses. There are 7 species of these viruses, 4 of which are very common. These nonspecific antibodies will interfere with assays for detection of COVID-19 specific antibodies. This is a common problem with antibody assays. For example, many actively used antibodies assays indicate as much as a 20% COVID-19 positive amongst the tested population even at the early stage of the pandemic. This percentage of positive testing is highly suspicious and indicative of high numbers of false positives.

Like any other pathogen infection, infection by COVID-19 virus provokes immune responses from the host. One of the immune responses is the production of antibodies, which specifically bind to antigens or unique conformations of antigens of the virus. These specific antibodies can be used to diagnose an infection of COVID-19 virus. The assays that detect these antibodies are commonly known as serological assays.

Serological assays appear to be particularly important for combating the COVID-19 pandemic. Molecular assays for detection of nucleic acids, while essential and highly sensitive, cannot meet all the needs. In addition, collecting and processing the samples pose significant risk to medical personnel. Moreover, a large number of infected individuals exhibit no or mild clinical symptoms and, therefore, may not seek medical treatment at the early phase of an infection. For the latter cases, serological assays are the only effective methods for detection of an infection.

However, there are several challenges with serological assays. Specific antibodies do not appear in serological samples normally until at least a week after an infection. More importantly, most adults have been infected with other coronaviruses, which may result in false positivity or reduced specificity, thereby interfering with a serological COVID-19 assay. False positivity is particularly problematic when the prevalence of COVID-19 infection is relatively low. For example, in a population with 5% prevalence of COVID-19 infection, a 5% false positive rate means that half of the reactive test results could be from false positives.

Moreover, molecular assays which detect viral nucleic acids may still produce false positive results. It is thus important to confirm the infection initially diagnosed with a molecular assay with a confirmatory serologic assay such as the one described herein.

The inventions herein describe include methods for more specifically detecting COVID-19 infection. In some embodiments, a COVID-19 serological assay detects and differentiates antibodies to at least two COVID-19 antigens. An infection of COVID-19 virus is confirmed when antibodies reactive to at least two different COVID-19 proteins or antigens are detected in the assay. Such assays can be used to further confirm the status of COVID-19 infection of samples that tested positive with in an initial screening by other serologic or molecular assays. The latter application is commonly known as confirmation of an infection.

Many people exhibit mild or no symptoms during the acute phase of a natural COVID-19 virus infection, which means that they may not be tested with a test such as a molecular or antigen test, which is commonly used for detection of acute infection, and consequently their natural infection status is unknown. Moreover, the population is being vaccinated with a vaccine targeting the S protein, which is the most used vaccine type. It is expected that some vaccinated individuals may still be infected with the virus, albeit exhibiting mild or no symptoms. Thus, the infection status of those who have mild, or no symptoms would have been unknown as they are not tested during acute phase of infection. It is likely that some of naturally infected individuals, even with mild or no symptoms during acute phase infection, may exhibit clinical symptoms long after acute infection, the so called “Long-Haul COVID-19” symptoms. It is therefore important to detect a natural infection, including mild and asymptomatic infection, among a population that is highly vaccinated.

Thus, in certain embodiments, a serological assay according to the methods can also be used to detect antibodies indicative of natural infection even if the sample is from an individual who has been vaccinated with a vaccine targeting the SARS-CoV-2 virus S protein, which is the commonly used vaccine type in combating COVID-19 disease.

### SUMMARY

The following enumerated paragraphs under this heading describe a few illustrative embodiments of the inventions herein described that exemplify some of their aspects and features. They are not exhaustive in illustrating their many aspects and embodiments, and are not in any way limitative of the inventions. Many other aspects, features and embodiments of the inventions are described herein and will be readily apparent to those of skill in the art upon reading the application and giving it due consideration in the full light of the prior art and the knowledge in the field.

The enumerated paragraphs under this heading are self-referential. The phrase “according to any of the foregoing or following” refers to all of the preceding and following enumerated paragraphs under this section, and their contents. All phrases, according to ## are direct references to the enumerated paragraph of that designation; e.g., “according to a46, means according to paragraph a46 in the enumerated paragraphs under this heading. All cross references of this type are combinatorial and are to be interpreted in same manner as they would be if they were multiple dependent claims (which they are not), except for redundancies and inconsistencies of scope.

The cross references under this heading, in particular, are used explicitly to provide a concise description showing the including of various combinations of the subject matter with one another. They are provided as a short hand way of compactly describing the many and various combination of aspects and particulars within the scope of the inventions herein described. Applicant reserves the right to expand any of these combinations individuals and multiply to spell them out in the specification or assert them in one or more claims as may be required or become desirable.

Ph1. A test strip, comprising:
a first separate and distinct area that indicates the presence or absence in a sample of antibodies to a first SARS-CoV-2 protein;
a second separate and distinct area that indicates the presence or absence in a sample of antibodies to a second SARS-CoV-2 protein different from the first protein;
a third separate and distinct area that indicates the positive or negative outcome of a positive control.

Ph2. A test strip according to any of the foregoing or the following, wherein
said first area has immobilized thereon at least one of the following proteins or an epitope-containing fragment thereof: a SARS-CoV-2 S protein, M protein, E protein or N protein;
said second area has immobilized thereon at least one of the following proteins or an epitope-containing fragment thereof: a SARS-CoV-2 S protein, M protein, E protein or N protein;
wherein said SARS-CoV-2 proteins or fragments immobilized on said first area are different from said SARS-CoV-2 proteins or fragments immobilized on said second area.

Ph3. A test strip according to any of the foregoing or the following, further comprising:
a fourth separate and distinct area having immobilized thereon at least one non-SARS-CoV-2 Coronavirus protein or epitope-containing fragment thereof.

Ph4. A test strip according to any of the foregoing or the following, further comprising:
a fifth separate and distinct area having therein in soluble form at least one non-SARS-CoV-2Coronavirus protein or epitope containing fragment thereof;
wherein the sample contacts said fifth area before contacting said first, second and third areas on the strip.

Ph5. A test strip according to any of the foregoing or the following, wherein the non-COVID-19 virus Coronavirus is one or more of Coronavirus OCE43, 299E, HKD1 and NL63.

Ph6. A test strip according to any of the foregoing or the following, further comprising:
a separate and distinct sample application area,
a separate and distinct conjugate contact area comprising labeled antibody binding reagents, wherein
sample applied to the sample application area contacts the conjugate contact area before contacting the first, second and third areas, whereby,
the labeled antibody binding reagents bind to antibodies in the sample, and the presence or absence of antibodies to SARS-CoV-2 is detected by the presence or absence of the detectable label binding to areas having immobilized thereon the SARS-CoV-2 proteins or fragments thereof.

Ph7. A test strip according to any of the foregoing or the following, wherein the label is colloidal gold or colored latex particles.

Ph8. A test strip according to according to any of the foregoing or the following, wherein the SARS-CoV-2 protein immobilized in the first area is the S protein or an epitope-containing fragment of the S protein.

Ph9. A test strip according to any of the foregoing or the following, wherein the SARS-CoV-2 protein immobilized in the second area is the E protein or an epitope-containing fragment of the E protein.

Ph10. A test strip according to any of the foregoing or the following, wherein the SAR-CoV-2 protein immobilized in the second area is the M protein or an epitope-containing fragment of the M protein

Ph11. A test strip according to any of the foregoing or the following, wherein the SAR-CoV-2 protein immobilized in the second area is the N protein or an epitope-containing fragment of the N protein

Ph12. A test strip according to any of the foregoing or the following, comprising:
a first separate and distinct area having thereon in soluble form one or more detectably labelled SARS-CoV-2 virus proteins or fragments thereof;
a second separate and distinct area having immobilized thereon an antibody binding agent that binds specifically to human IgG antibodies;
a third separate and distinct area having immobilized thereon an antibody binding agent that binds specifically to human IgM antibodies;
a fourth separate and distinct area the indicates the positive or negative outcome of a positive control;
wherein a sample applied to the test contacts the first area before contacting the second and third areas,
whereby anti-SARS-CoV-2 IgG antibodies in the sample that bind to detectably labeled SARS-CoV-2 proteins in the first area are indicated by detectable label binding to the second area and
anti-SARS-CoV-2 IgM antibodies in the sample that bind to detectably labeled SARS-CoV-2 proteins in the first area are indicated by detectable label binding to the second area.

Ph13. A test strip according to any of the foregoing or the following, wherein the first area further comprises proteins of a non-SARS-CoV-2 Coronavirus that are not detectably labeled.

Ph14. A device for detecting antibodies to SARS- CoV-2 in a sample, comprising,
at least one test strip according to any of the foregoing or the following;
a port for applying a sample to a sample application area on the strip;
a source of buffer disposed upstream of the sample application;
and windows for detecting the presence or absence of detectable label binding to the SARS-CoV-2 and/or non-SARS-CoV-2 Coronavirus proteins and/or fragments immobilized in the respective separate and distinct areas thereon.

Ph15. A device for detecting anti-SARS-CoV-2 antibodies in a sample, comprising:
at least one test strip according to any of the foregoing or the following,
a port for applying a sample to a sample application area on the strip;
a source of buffer disposed upstream of the sample application;
and windows for detecting the presence or absence of detectable label binding to the anti-IgG and anti-IgM antibodies immobilized in the respective separate and distinct areas thereon.

Ph16. A device according to any of the foregoing or the following, wherein the buffer pH is 9.5 to 11.5.

Ph17. A kit comprising
a device according to any of the foregoing or the following, and
a buffer solution of pH 9.5-11.5.

Ph18. A method for determining the status of infection by SARS-CoV-2 virus in a subject vaccinated with a vaccine targeting the SARS-CoV-2 S protein or an epitope containing fragment of the S protein, comprising:

contacting an antibody-containing sample from a patient with a test strip comprising:
a first separate and distinct area that indicates the presence or absence in a sample of antibodies to a first SARS-CoV-2 protein, wherein said first protein is targeted by a SARS-CoV-2 vaccine with which the subject has been inoculated;
a second separate and distinct area that indicates the presence or absence in a sample of antibodies to a second SARS-CoV-2 protein different from the first protein, wherein said second protein is not targeted by a SARS-CoV-2 vaccine with which the subject has been inoculated; and
a third separate and distinct area that is a control indicating whether or not the test functioned properly;

determining from said third area that the test functioned properly;

determining binding of antibodies in said sample to said first and said second area;

wherein binding to said first area relative to binding to said second area is indicative of the presence or absence of infection by SARS-CoV-2;

wherein binding to said second area relative to said first area exceeding a threshold value indicates the presence of infection by SARS-CoV-2, and below that value indicates an absence of infection by SARS-CoV-2.

Ph19. A method according to any of the foregoing or the following, wherein
said first area has immobilized thereon at least one of the following proteins or an epitope-containing fragment thereof: a SARS-CoV-2 S protein, M protein, E protein or N protein, wherein said one or more proteins is targeted by a SARS-CoV-2 vaccine to which the subject has been exposed;
said second area has immobilized thereon at least one of the following proteins or an epitope-containing fragment thereof: a SARS-CoV-2 S protein, M protein, E protein or N protein, wherein said one or more proteins is not targeted by a SARS-CoV-2 vaccine to which the subject has been exposed.

Ph 20. A method according to any of the foregoing or the following, wherein
said first area has immobilized thereon the SARS-CoV-2 S protein or an epitope-containing fragment of the S protein, and
said second area has immobilized thereon a SARS-CoV-2 N protein, or an epitope-containing fragment thereof.

Ph 21. A method according to any of the foregoing or the following comprising contacting an antibody-containing sample from a subject to a test strip according to any of the foregoing.

Ph 22. A method according to any of the foregoing or the following comprising contacting an antibody-containing sample from a subject to device comprising a test strip according to any of the foregoing.

Ph23. A method according to any of the foregoing or the following, wherein binding of antibodies in the same to said proteins or epitope containing fragments is carried out a pH of 9.5 to 11.5.

### BRIEF DESCRIPTION OF THE FIGURES

FIG. 1 is a diagram depicting the detection principle of one embodiment. The sample contains antibodies to both COVID-19 and non COVID-19 coronavirus.

FIG. 2 is a diagram depicting the same detection principle as described in FIG. 1. The sample contains antibodies to two antigens of COVID-19 virus and no antibodies to non COVID-19 coronavirus. Thus, the sample is positive for COVID-19 infection but negative for non COVID-19 viruses.

FIG. 3 is diagram depicting the same detection principle as described in FIG. 1. The sample contains no antibodies to any antigen of COVID-19 virus but contains antibodies to non COVID-19 coronavirus. Thus, the sample is negative for COVID-19 infection but positive for non COVID-19 virus infection.

FIG. 4 is a diagram depicting the same detection principle as described in FIG. 1. This sample showed reactivity to only one COVID-19 protein (the S protein) and no reactivity to the non COVID-19 N protein, indicating that the sample contains no antibodies to non COVID-19 coronavirus. The status of COVID-19 infection is indeterminate as there is only one antigen showing reactivity.

FIG. 5 is a diagram depicting the same detection principle as described in FIG. 1. This sample showed reactivity to only one COVID-19 protein (the N protein) and reactivity to the non COVID-19 N protein, indicating that the sample contains antibodies to non COVID-19 coronavirus. The status of COVID-19 infection is indeterminate as there is only one antigen showing reactivity. The reactivity to COVID-19 N protein could be due to high titer antibodies to non COVID-19 N proteins.

FIG. 6 is diagram depicting the same detection principle as described in FIG. 1. This sample showed no reactivity to any COVID-19 protein or to the non COVID-19 virus N protein, indicating that the sample contains no antibodies to either COVID-19 coronavirus or non COVID-19 coronavirus.

FIG. 7 is a diagram depicting one embodiment of the invention. Two test strips are configured into one test as described in FIG. 1. This sample showed reactivity only to COVID-19 S protein. This test result, with or without reactivity to non COVID-19 coronavirus antigen, may indicate antibody response from vaccination if the sample donor has been vaccinated.

FIG. 8 is a diagram depicting the same detection principle. In this example, the test is a single test strip in a lateral flow chromatographic format. This embodiment of the invention is for detection of COVID-19 virus specific IgG and IgM antibodies. In this example, the IgG and IgM lines are coated with specific antibodies which can capture human IgG and IgM antibodies, respectively. The conjugate pad contains gold particles coated with COVID-19 virus proteins, S and N, or their derivatives. The sample pad contains a mixture of S and N proteins derived from non COVID-19 virus strains OC43, 299E, HKD1 and NL63.

FIG. 9 illustrates an example of a qSARS-CoV-2 IgG/IgM Rapid Test device.

FIG. 10 shows a lateral flow assay for binding to SARS-CoV-2 N and S proteins for detection of natural infection even if the sample is from an individual who is vaccinated with an S-protein based vaccines, as described in Example 7.

### GLOSSARY

As used herein certain terms have the meanings set forth below.

“A” and “an” both mean one or more; at least one.

COVID-19—is used herein to denote both the SARS-CoV-2 virus and the disease it causes.

SARS-CoV-2 is the virus that causes COVID-19 disease. SARS-CoV-2 is an acronym for “severe acute respiratory syndrome coronavirus 2”. The SARS-CoV-2 virus is also referred to herein as COVID-19. SARS-CoV-2 is a coronavirus. A variety of other Coronaviruses are known that do not cause COVID-19 disease. Several are common in humans. A few examples of non-SARS-CoV-2 Coronaviruses are the OC43, 299E, HKD1 and NL63 Coronavirus.

SARS-CoV-2 is an enveloped virus consisting of a positive-sense, single-stranded RNA genome of around 30 kb. Two overlapping ORFs, ORF1a and ORF1b, are translated from the positive-strand genomic RNA and generate continuous polypeptides, which are cleaved into a total of 16 nonstructural proteins (NSPs). The translation of ORF1b is mediated by a −1 frameshift that allows translation to continue beyond the stop codon of ORF1a. Negative-strand RNA intermediates are produced from the viral genome and serve as templates for the synthesis of positive-strand genomic RNA and subgenomic RNAs. The different subgenomic RNAs encode four conserved structural proteins—spike (S), envelope (E), membrane (M) and nucleocapsid (N). See for instance, Finkel et al., Nature 589, 125-130 (2021), which is incorporated by reference herein in its entirety.

### DESCRIPTION

The inventions herein describe include methods for more specifically detecting COVID-19 infection. In some embodiments, a COVID-19 serological assay detects and differentiates antibodies to at least two COVID-19 antigens. An infection of COVID-19 virus is confirmed when antibodies reactive to at least two different COVID-19 proteins or antigens are detected in the assay. Such assays can be used to further confirm the status of COVID-19 infection of samples that tested positive with in an initial screening by other serologic or molecular assays. The latter application is commonly known as confirmation of an infection.

The inventions herein described also include a method for detection of and differentiation between COVID-19 virus infection and non COVID-19 coronavirus infection.

A combination of detection of two or more COVID-19 virus antigens and detection of and differentiation between COVID-19 virus and non COVID-19 coronavirus infection may be used to further improve the accuracy of a serological assay.

The inventions herein described also include a method for reducing false positive rate of a COVID-19 serologic assay due to cross reactivity of antibodies to non COVID-19 coronaviruses or other viruses.

Vaccines have been and are continuing to be developed for the COVID-19 virus. Most of the vaccines and vaccine candidates target the COVID-19 S protein. Another application of the present invention is for detection of natural infection status among vaccinated population. Many vaccinated or unvaccinated individuals who exhibit mild or no symptoms may not know their status of natural infection. It is important to identify those who do not know their natural infection status. As more and more individuals are vaccinated with vaccines targeting the S protein, which would generate antibody response to the S protein, a test is needed for detection of natural infection among those who have been vaccinated with this type of vaccine. The assays in certain embodiments disclosed herein may also be used to detect natural infection among vaccinated populations.

Some COVID-19 assays described herein can be used to distinguish acute infections from convalescent infections, the latter being recovered patients who are in theory immune or have some level of immunity to a second infection of COVID-19. IgM antibodies become detectable around day 10 of an infection, and IgG antibodies become detectable after IgM antibodies, generally few days later. From 1 to 5 months later—for example, around 3 months later—the amount of detectable IgG antibodies diminishes. IgM antibodies becomes undetectable much sooner, for example, about 1 to 2 months, in many cases within 45 days to 60 days after an infection.

In view of the above, one strategy is to develop assays which can detect and differentiate IgM from IgG. Such an assay must

1) detect both IgG and IgM, and

2) tell IgM and IgG apart.

However, a problem with prior art assays with such a strategy arises, for example, when only clinical specimens are used to validate results. Clinical specimens, particularly those from patients who have active infections, for example, from about day 10 to about day 40, usually contain both IgG and IgM. A reactive IgM test result means the person still has recent infection and possibly is still infectious. However, the prior art assays are actually detecting cross reactivity of IgG not just IgM, as IgG is much more abundant than IgM. This often results in a false positive being indicated. That is, the test result is mistaking cross reactivity of IgG for reactive IgM.

The above has extreme real-world consequences. If a person is only IgG positive, the interpretation would be that this person may have been exposed to the virus a distant time ago and is now safe and noninfectious. While a reactive IgM test result would be interpreted as an active infection and contagious person where the person and all recent contacts need to be immediately quarantined for an extended period of time. This is why IgG-IgM cross reactivity can be disastrous and very common in the prior art testing.

The COVID-19 virus encodes functional and non-functional or structural proteins. Because of the abundance of structural proteins, they are commonly used as antigens in a serological assay. COVID-19 virus encodes four structural proteins, including spike (S) glycoprotein, envelope (E) protein, membrane (NI) protein, and nucleocapsid (N) protein. The S protein usually is further divided into subunits S1 and S2, where S1 binds the host cell receptor and S2 promotes membrane fusion with the host cells. In one embodiment, a serological assay disclosed herein is able to detect and differentiate antibodies against at least two of the following proteins, or parts of these proteins, as antigens: S (or S1 and S2 separately), E, M and N protein.

In other embodiments, an antigen or a mixture of antigens from non COVID-19 coronavirus are also used along with COVID-19 viral proteins, thereby enabling detection and differentiation of COVID-19 infection from non COVID-19 coronaviral infection. For example, COVID-19 viral nucleocapsid protein (N protein) and non COVID-19 N protein

A number of assay formats can be used in assays described herein. These formats include, but are not limited to, lateral flow chromatography, ELISA or EIA, microarray, fluorescent beads based multiplexing assays or any other multiplexing immunoassay format. In these multiplexing formats, antibodies reactive to different antigens can be simultaneously detected and differentiated.

Some embodiments of inventions herein described use quality control specimens that are unequivocal for IgG or IgM antibodies. Some embodiments use neutralizing antibodies. For example, in one embodiment mouse antibodies are created. Then the Fab fragment (the antigen binding fragment) is inserted into human antibodies, IgG or IgM or others to create humanized antibodies. In some embodiments, these humanized antibodies are screened. They do not have to be neutralizing. However, they do need to react with antigens in the assay. These are used for QC materials and the assays of some embodiments disclosed herein.

In some embodiments, adjusting the pH to outside the normal pH for an antibody/antigen assay results in great improvement of the specificity of the assay, for example, by using a pH of 10 or 11. These conditions are generally considered unfavorable. However, it has been found that under these conditions—for example, a high pH of 10 to 11, only highly specific antibodies are detected, and the number of false positive results is greatly reduced. Embodiments in this regard have the additional advantage of being a low-cost solution to the problem of false positive results. pH is only one of the conditions which can be adjusted to increase the specificity and reduce false positive results.

Some embodiments create and utilize an extensive and optimized set of controls. For instance, controls in some embodiments include the following: IgG positive (negative human blood sample spiked with humanized IgG), IgM positive (negative human blood sample spiked with humanized IgM) and a negative sample. Such controls allow for a great improvement in the performance of COVID-19 assays by increasing specificity and decreasing false positive test results.

Certain embodiments of methods herein disclosed are illustrated in the Figures.

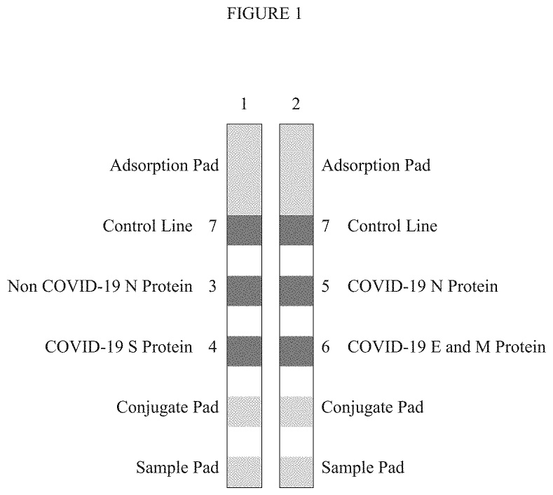

FIG. 1 depicts a method using a lateral flow chromatographic method. In this embodiment, the assay is capable of detecting antibodies against 1) Non COVID-19 coronavirus nucleocapsid protein (N protein), 2) COVID-19 coronavirus nucleocapsid protein (N protein), 3) COVID-19 coronavirus S protein, and 4) COVID-19 coronavirus envelope and/or membrane protein.

Here the non COVID-19 coronavirus strains are referring to OC43, 299E, HKD1 or NL63 strains, which do not include SARS (severe acute respiratory syndrome) or MERS (middle east respiratory syndrome).

In one embodiment, the lateral flow chromatographic assay comprises two nitrocellulose membrane strips 1 and 2 in order to increase the number of antigens being detected. Each strip contains three detection lines consisting of a control line and two antigen lines. In this embodiment, there are a total of four antigen lines.

In one embodiment, the antigen line 3 is for detection of non COVID-19 coronavirus infection. In some embodiment, the antigen line 3 comprises N protein or a portion of the N protein derived from non COVID-19 coronavirus. The non COVID-19 coronavirus N protein is derived from coronavirus strains OC43, 299E, HKD1 or NL63. To detect infection of all four non COVID-19 virus strain, a mixture of N proteins, or portions of the proteins, derived from each of OC43, 299E, HKD1 and NL63 strain may be used.

FIG. 1 shows a diagram depicting an embodiment in which two test strips are configured into one test. Antigens are coated onto various positions as indicated. The conjugate pad contains protein A coated gold particles. The sample pad in strip 1 is coated with COVID-19 N protein while the sample pad in strip 2 is coated with non COVID-19 N protein. Same amount of sample is added to strips 1 and 2, followed by addition of sample buffer. This sample showed reactivity to at least two COVID-19 proteins and the non COVID-19 N protein, indicating that the sample contains antibodies to both COVID-19 virus and to non COVID-19 coronavirus.

Referring to FIG. 1, antigen line 4 is an antigen derived from the S protein of COVID-19 virus. The S protein can be a full-length protein or a partial protein so long as it is derived from the amino acid sequence of COVID-19 virus.

Referring to FIG. 1, antigen line 5 is an antigen derived from the N protein of COVID-19 virus. The N protein can be a full-length protein or a partial protein so long as it is derived from the amino acid sequence of COVID-19 virus.

Referring to FIG. 1, antigen line 6 is an antigen derived from the M protein or E protein of COVID-19 virus. The M or E protein can be a full-length protein, a partial protein or a peptide containing an epitope so long as they are derived from the amino acid sequence of COVID-19 virus. A mixture of M and E proteins or peptides derived from these proteins can be used in this antigen line.

Other than the antigen lines, each strip contains a control line 7. Control line may be a line coated with an entity that can capture the gold particles coated with antigens or a generic molecule such as protein A, which can detect captured antibodies on the antigen lines.

In a lateral flow chromatography assay, the nitrocellulose strip normally contains three additional parts: an adsorption pad, which helps drive the capillary movement of liquid solutions, a conjugate pad, which is coated with gold particles or other detectable components coated with antibodies, antigens or other antibody binding molecules such as protein A. The conjugates in the conjugate pad enables detection of specific antibodies captured onto the antigen lines.

A lateral flow chromatography assay strip normally also contains sample pad, which contains various ingredients that help dissolve the reagents in the conjugate pad and promote specific binding of antibodies to antigens. In certain embodiments, a protein or a mixture of proteins derived from non COVID-19 coronavirus is added to the sample pad to increase the specificity of detection for COVID-19 virus infection. Conversely, a protein or a mixture of proteins derived from COVID-19 coronavirus is added to the sample pad to increase the specificity of detection for non COVID-19 virus infection.

Many variations can be configured with various detection platforms to achieve detection and differentiation of infections with COVID-19 or non COVID-19 coronavirus, and detection and differentiation of two or more antigens derived from COVID-19 virus.

In one variation, the assay is designed for detection of COVID-19 infection only. According to an aspect of the invention, proteins from non COVID-19 coronaviruses are used to reduced potential cross reactivity with COVID-19 antigens in a test with non COVID-19 virus antigens. In a lateral flow format, the non COVID-19 proteins can be added to the sample pad or to sample buffer to neutralize the antibodies against non COVID-19 proteins thereby reducing the potential cross reactivity to COVID-19 antigens. For example, if COVID-19 virus S and N proteins, or the derivatives of these proteins, are used as antigens for detection of COVID-19 specific antibodies, non COVID-19 coronavirus S and N proteins may be used in the assay to reduce nonspecific cross reactivity, thereby increasing the assay specificity.

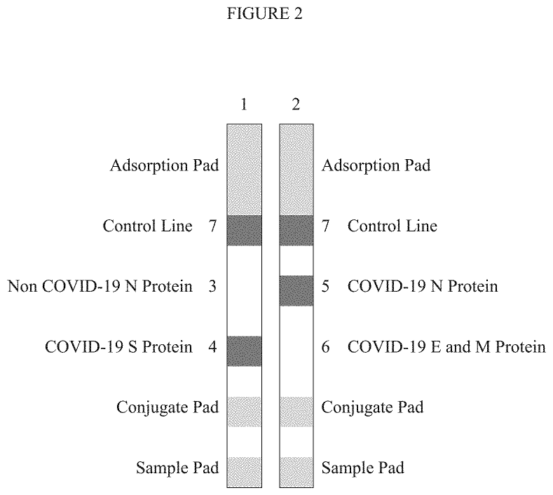

FIG. 2 shows a diagram depicting an embodiment in which two test strips are configured into one test as depicted in FIG. 1. This sample showed reactivity to two COVID-19 proteins and no reactivity to the non COV1D-19 N protein, indicating that the sample contains antibodies to COVID-19 virus but no antibodies to non COVID-19 coronavirus.

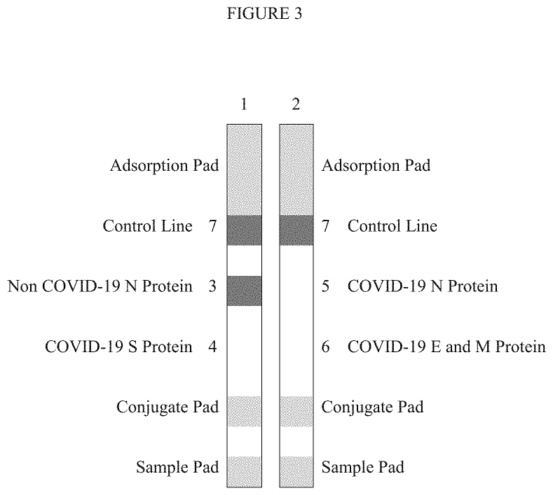

FIG. 3 shows a diagram depicting an embodiment in which two test strips are configured into one test as described in FIG. 1. This sample showed reactivity to only to non COVID-19 N protein, indicating that the sample contains antibodies to non COVID-19 coronavirus only.

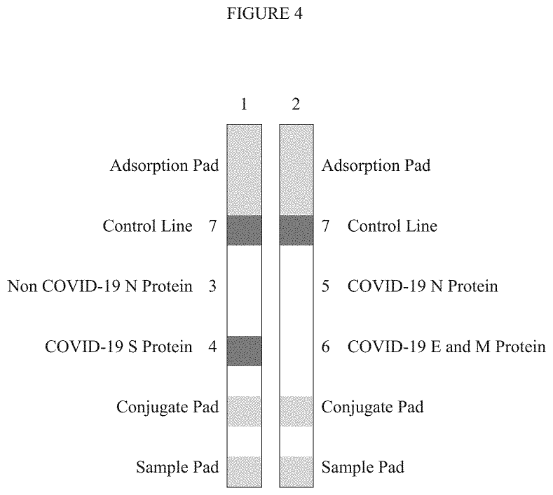

FIG. 4 shows a diagram depicting an embodiment in which two test strips are configured into one test as described in FIG. 1. This sample showed reactivity to only one COVID-19 protein (the S protein) and no reactivity to the non COVID-19 N protein, indicating that the sample contains no antibodies to non COVID-19 coronavirus. The status of COVID-19 infection is indeterminate as there is only one antigen showing reactivity.

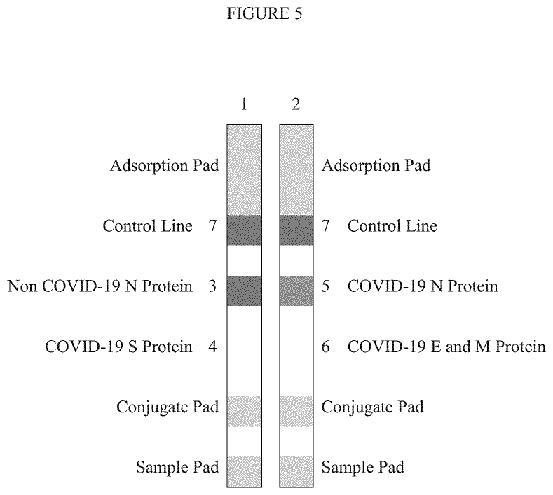

FIG. 5 shows a diagram depicting an embodiment in which two test strips are configured into one test as described in FIG. 1. This sample showed reactivity to only one COVID-19 protein (the N protein) and reactivity to the non COVID-19 N protein, indicating that the sample contains antibodies to non COVID-19 coronavirus. The status of COVID-19 infection is indeterminate as there is only one antigen showing reactivity. The reactivity to COVID-19 N protein could be due to high titer antibodies to non COVID-19 N proteins.

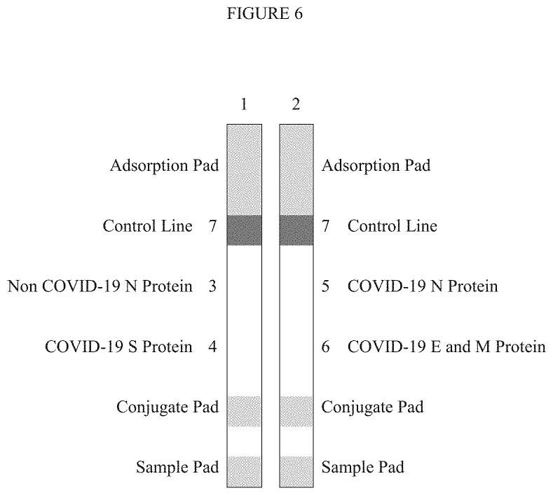

FIG. 6 shows a diagram depicting an embodiment in which two test strips are configured into one test as described in FIG. 1. This sample showed no reactivity to any COVID-19 protein or to the non COVID-19 N protein, indicating that the sample contains no antibodies to either COVID-19 coronavirus or non COVID-19 coronavirus.

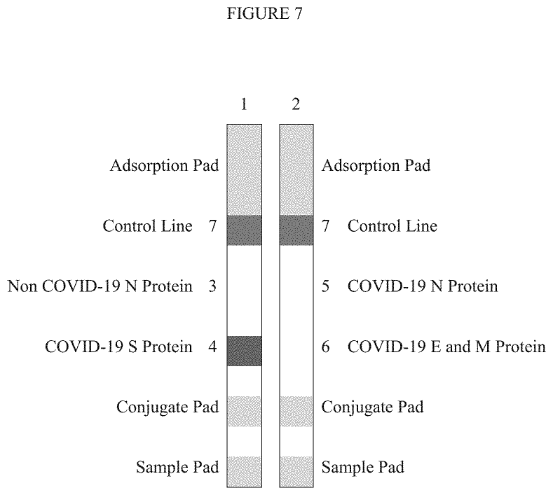

FIG. 7 shows a diagram depicting an embodiment in which two test strips are configured into one test as described in FIG. 1. This sample showed reactivity only to COVID-19 S protein. This test result, with or without reactivity to non COVID-19 coronavirus antigen, may indicate antibody response from vaccination if the sample donor has been vaccinated.

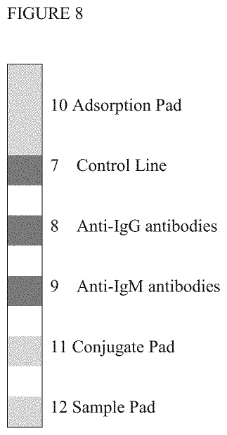

FIG. 8 shows a diagram depicting an embodiment for detection of COVID-19 virus specific IgG and IgM antibodies. In this example, the IgG 8 and IgM 9 lines are coated with specific antibodies against human IgG and IgM antibodies, respectively. The conjugate pad 11 contains gold particles coated with COVID-19 virus proteins, S and N, or their derivatives. The sample pad 12 contains a mixture of S and N proteins derived from non COVID-19 virus strains OC43, 299E, HKD1 and NL63.

In some embodiments, the invention includes a qSARS-CoV-2 IgG/IgM Rapid Test.

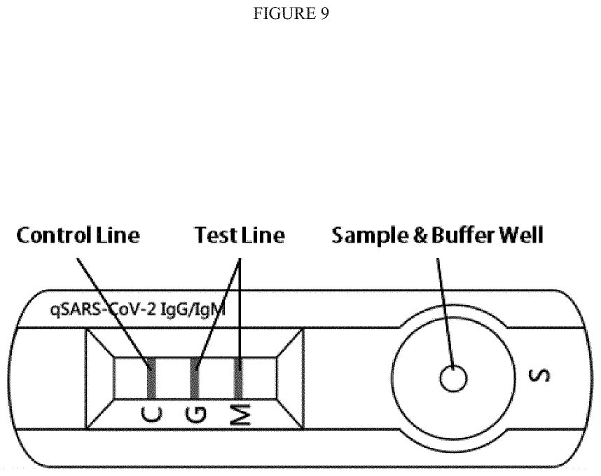

FIG. 9 shows an example of a qSARS-CoV-2 IgG/IgM Rapid Test device. In some embodiments, this test is a lateral flow immunoassay intended for the qualitative detection and differentiation of IgM and IgG antibodies to SARS-CoV-2 in serum, plasma (EDTA, citrate) or venipuncture whole blood specimens from patients suspected of COVID-19 infection by a healthcare provider. The qSARS-CoV-2 IgG/IgM Rapid Test is an aid in the diagnosis of patients with suspected SARS-CoV-2 infection in conjunction with clinical presentation and the results of other laboratory tests.

The test is a lateral flow chromatographic immunoassay which can detect antibodies against the SARS-CoV-2 virus. The test cassette includes:

1) a burgundy colored conjugate pad containing SARS-CoV-2 recombinant antigens (S and N proteins) conjugated with colloidal gold (SARS-CoV-2 conjugates) and rabbit IgG-gold conjugates;

2) a nitrocellulose membrane strip containing an IgG line (G Line) coated with anti-human IgG, an IgM line (M Line) coated with anti-human IgM, and the control line (C Line) coated with goat anti-rabbit IgG.

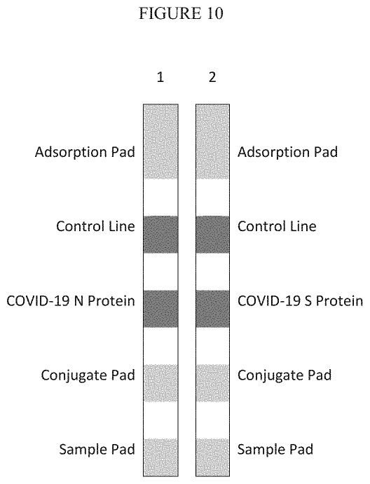

FIG. 10 shows a lateral flow assay for binding to SARS-CoV-2 N and S proteins to detect natural infection even if the sample is from an individual who is vaccinated with an S-protein based vaccines, as described in Example 7.

### EXAMPLES

The inventions herein disclosed are additionally described by the following Examples. These Examples are exclusively illustrative of particular aspects and embodiments and are not in any way limitative.

### Example 1

### A SARS-CoV-2 Serological Test

Using the test device of FIG. 9 as an example, when a correct volume of test specimen is dispensed into the sample well of the test cassette, the specimen migrates by capillary action along the cassette. The anti-SARS-CoV-2 virus IgG, if present in the specimen, will bind to the SARS-CoV-2 conjugates. If IgG is present in the specimen, the immunocomplex will then captured by the anti-human IgG line, forming a burgundy colored G Line, indicating a SARS-CoV-2 virus IgG positive test result.

The anti-SARS-CoV-2 virus IgM, if present in the specimen, will bind to the SARS-CoV-2 conjugates. The immunocomplex is then captured by the anti-human IgM line, forming a burgundy colored M Line, indicating a SARS-CoV-2 virus IgM positive test result. Information regarding the immune response to SARS-CoV-2 is limited and still evolving. The test contains an internal control (C Line) which should exhibit a burgundy colored band of goat anti-rabbit IgG/rabbit IgG-gold conjugate immunocomplex regardless of the color development on any of the test bands (G and M Lines). If no control band is observed, the test result is invalid and the specimen must be retested.

### Example 2

### Some Test Kits and Testing Procedures

Three different embodiments of test kits are set out in Table 1 below:

TABLE 1
Composition of Test Kits

| Catalog # | | 5515C025 | 5515C050 | 5515C100 |
|---|---|---|---|---|
| Kit Size (# of Tests) | | 25 | 50 | 100 |
| Components | Test Cassette (#) | 25 | 50 | 100 |
| | Sample Diluent | 1 | 1 | 1 |
| | (# of Bottles) | | | |
| | Transfer pipette (#)* | 25 | 50 | 100 |
| | IFU Leaflet | 1 | 1 | 1 |

*Transfer pipette is packaged inside the test cassette pouch.

A control set including a positive and a negative control may be provided in kit. A vial of positive or negative control contains approximately 40 microliters of specimens. In some embodiments each control vial is sufficient for conducting 3 tests.

Materials
Conjugate Pad: SARS-CoV-2 antigen coated gold particles
G Line: Anti-human IgG
M Line: Anti-human IgM
C Line: Goat anti-rabbit IgG
Sample Buffer: 0.01M PBS; PH 7.4
Negative Control: Negative human serum, chemically inactivated.
Positive Control: Negative human serum spiked with positive serum, chemically inactivated. It may be reactive to the IgM line, IgG line or both.

Other Materials
Timer

Storage and Stability
1. Store the detector buffer at 2-30° C. The buffer is stable up to 12 months.
2. Store the qSARS-CoV-2 IgG/IgM Rapid Test at 2-30° C.; its shelf life is up to 12 months.
3. If stored at 2-8° C., ensure that the test device is brought to 15-30° C. before opening.
4. Do not freeze the kit or store the kit over 30° C.

Specimen Collection and Preparation
Consider any materials of human origin as infectious and handle using standard biosafety procedures.

Plasma
1. Collect blood specimen into a lavender or blue top collection tube (containing EDTA or citrate, respectively, in a Vacutainer®) by venipuncture.
2. Separate the plasma by centrifugation.
3. Carefully withdraw the plasma into a new pre-labeled tube.

Serum
1. Collect blood specimen into a red top collection tube (containing no anticoagulants in a Vacutainer®) by venipuncture.
2. Allow the blood to clot.
3. Separate the serum by centrifugation.
4. Carefully withdraw the serum into a new pre-labeled tube.

For Serum and Plasma Stability
Test specimens as soon as possible after collection.
If specimens are not tested immediately, store at 2-8° C. for up to 3 days. The specimens should be frozen at −20° C. for longer storage.
For frozen samples, avoid more than 4 freeze-thaw cycles. Prior to testing, bring frozen specimens to room temperature slowly and mix gently.
Specimens containing visible particulate matter should be clarified by centrifugation before testing.
Samples demonstrating gross lipemia, gross hemolysis or turbidity should not be used in order to avoid interference on result interpretation.

Whole Blood
1. Drops of whole blood can be obtained by venipuncture.
2. Whole blood specimens should be stored at 2-8° C. if not tested immediately. The specimens should be tested within 24 hours of collection.

Test Procedure
Step 1: For fresh samples, begin with Step 2. For frozen samples, bring the specimens and test components to room temperature, and mix the specimen well once thawed.
Step 2: When ready to test, open the pouch at the notch and remove the test device. Place the test device on a clean, flat surface.
Step 3: Label the device with specimen ID number.
Step 4: Using a transfer pipette, transfer serum, plasma or whole blood, careful not to exceed the specimen well. The volume of the specimen is around 10 μL. For better precision, transfer specimen by a pipette capable of delivering 10 μL of volume.
Holding the transfer pipette vertically, dispense 10 μL of the specimen into the center of the sample well (S well) making sure that there are no air bubbles.
Then, add 2 drops of Sample Diluent immediately into the sample well (S well).
Step 5: Set a timer.
Step 6: Read the results in 15-20 minutes.

Quality Control
1. Internal Control: This test contains a built-in control feature, the C Line. The C Line develops after addition of the specimen and sample diluent. If the C Line does not develop, the test is invalid.
2. Positive and Negative Control: Positive and negative controls should be tested to ensure the proper performance of the assay, particularly under the following circumstances:
   A. A new operator uses the kit;
   B. A new lot of test kits is used;
   C. A new shipment of kits is used;
   D. The temperature used during storage of the kit falls outside of 2-30° C.;
   E. The temperature of the test area falls outside of 15-30° C.;
   F. To verify a higher than expected frequency of positive or negative results;
   G. To investigate the cause of repeated invalid results; or
   H. A new test environment is used (e.g., natural light vs. artificial light).
In some embodiments, the positive and negative controls should be spun down before use. When performed properly, in addition to the presence of C Line, no line should be visible for the negative control and the G Line or M Line or both lines is/are visible for the positive controls. The positive control may contain IG or IgM or both analytes. Additional controls may be qualified and tested by the user.

Interpretation of the Assay Result

Valid Assay

1.1 In addition to the presence of the C Line, if only the G Line is developed, the test result indicates the presence of IgG anti-SARS-CoV-2 virus. The result is IgG positive or reactive, consistent with a recent or previous infection.
1.2 In addition to the presence of the C Line, if only the M Line is developed, the test indicates the presence of IgM anti-SARS-CoV-2 virus. The result is IgM positive or reactive, consistent with an acute or recent SARS-CoV-2 virus infection.
1.3 In addition to the presence of the C Line, if both G and M Lines are developed, the test indicates the presence of IgG and IgM anti-SARS-CoV-2 virus. The result is IgG and IgM positive or reactive, suggesting current or recent SARS-CoV-2 virus infection.

Invalid Assay
If the C Line does not develop, the assay is invalid regardless of color development of the G or M Lines as indicated below. Repeat the assay with a new device.

### Example 3

### Testing of RT-PCR Positive Clinical Specimens

Ninety-eight (98) positive serum or plasma samples collected from individuals who tested positive with a RT-PCR method for SARS-CoV-2 infection and were quarantined in a makeshift hospital were used in this study. These patients, at the time of sample collection, exhibited mild or no clinical symptoms. These samples, along with 180 negative serum or plasma samples collected prior to September 2019, were coded and tested together with the qSARS-CoV-2 IgG/IgM Rapid Test. Of the 98 positive samples, ninety-one (91) were tested positive with IgG or IgM or both. Of the 180 negative samples, one hundred seventy-four (174) were tested negative.

Another 30 samples were collected from hospitalized individuals who were clinically confirmed positive for SARS-CoV-2 infection and exhibited severe symptoms. These samples, along with 70 negative serum or plasma samples collected prior to September 2019, were coded and tested together with the qSARS-CoV-2 IgG/IgM Rapid Test. Of the 30 positive samples, twenty-nine (29) were tested positive with IgG or IgM or both. Of the 70 negative samples, sixty-five (65) tested negative. The day of collection relative to the onset of illness was unknown.

Taken together, the qSARS-CoV-2 IgG/IgM Rapid Test had a Positive Percent Agreement and Negative Percent Agreement of 93.75% (95% CI: 88.06-97.26%) and 96.40% (95% CI: 92.26-97.78%), respectively.

Results are shown in Table 2 below.

TABLE 2
Performance Characteristics of a Test Kit

| | | | Comparator | | |
|---|---|---|---|---|---|
| | | | Pos | Neg | Subtotal |
| qSARS- | Pos | IgG+/IgM+ | 62 | 0 | 62 |
| CoV-2 | | IgG−/IgM+ | 43 | 4 | 47 |
| IgG/IgM | | IgG+/IgM− | 15 | 6 | 21 |
| Rapid | | | | | |
| Test | Neg | IgG−/IgM− | 8 | 240 | 248 |
| Subtotal | | | 128 | 250 | 378 |

Positive Percent Agreement (PPA) = 120/128 (93.8%), 95% CI: 88.2% to 96.8%.
Negative Percent Agreement (NPA) = 240/250 (96.0%), 95% CI: 92.8% to 97.8%.

### Example 4

### Venous Whole Blood Specimens Spiked with Positive Samples

Fifty (50) negative whole blood samples were spiked with positive serum at 1:100. Another fifty (50) whole blood specimens were spiked with negative serum at the same dilution. These 100 specimens were coded and tested with the qSARS-CoV-2 IgG/IgM Rapid Test. All spiked samples were correctly identified by the test except for one of the negative samples, which was tested positive with the test. Thus, there was a 99% concordance rate with expected results when venous whole blood specimens are used.

### Example 5

### No Cross Reactivity Was Observed With Other Viruses

Cross-reactivity of the qSARS-CoV-2 lgG/IgM Rapid Test was evaluated using serum or plasma samples which contain antibodies to the pathogens listed below. No false positivity or false negativity was found with any of the following.

Human coronavirus panel

HBV

HCV

HIV-1

HIV-2

Adenovirus

Human Metapneumovirus (hMPV)

Parainfluenza virus 1-4

Influenza A

Influenza B

Enterovirus 71

Respiratory syncytial virus

Rhinovirus

Chlamydia pneumoniae

Streptococcus pneumoniae

Mycobacterium tuberculosis

Mycoplasma pneumoniae

EB Virus

### Example 6

### Endogenous Substances Do Not Interfere

Low titer SARS-CoV-2 antibody positive serum samples and SARS-CoV-2 antibody negative serum samples were spiked with one of the following substances to specified concentrations and tested in multiple replicates. No false positivity or false negativity was found with the examples in Table 3 below.

TABLE 3

| | | |
|---|---|---|
| Hemoglobin - | 10 | mg/mL |
| Bilirubin Conjugated - | 0.4 | mg/mL |
| Bilirubin Unconjugated - | 0.4 | mg/mL |
| Triglycerides - | 15 | mg/mL |
| Cholesterol - | 4 | mg/mL |
| Human Anti-mouse Antibody (HAMA) - | 800 | ng/mL |
| Rheumatoid Factor - | 2000 | IU/mL |
| Human Serum Albumin - | 60 | mg/mL |
| Histamine hydrochloride - | 4 | mg/L |
| α-IFN - | 200 | mg/L |
| Zanamivir - | 1 | mg/L |
| Oseltamivir carboxylate - | 1 | mg/L |
| Abidol - | 40 | mg/L |
| Levofloxacin - | 200 | mg/L |
| Ceftriaxone - | 400 | mg/L |
| Meropenem - | 200 | mg/L |
| Tobramycin - | 10 | mg/L |
| Ribavirin - | 40 | mg/L |
| Human IgG - | 8 | mg/mL |
| Human IgM | 0.4 | mg/mL |

### Example 7

### Detection of Natural Infection Using a Sample from an Individual Who Is Vaccinated with a Vaccine Targeting the S Protein

Many people exhibit mild or no symptoms during the acute phase of a natural COVID-19 virus infection, which means that they are not tested with a test such as a molecular or antigen test, which is commonly used for detection of acute infection. Moreover, the population is being vaccinated. It is expected that some vaccinated individuals may still be infected with the virus, albeit exhibiting mild or no symptoms. It is therefore important to detect a natural infection among a population that is highly vaccinated with vaccines targeting the S protein.

The qSARS-CoV-2 NS Antibody Rapid Test described herein is a lateral flow-based test that detects and differentiates total antibodies against the SARS-CoV-2 N and S proteins and is useful to distinguish antibodies generated by infection with SARS-CoV-2 from those generated in response to immunization with a protein S-based vaccine. The test strip of this Example is illustrated in FIG. 10.

The test in this example can detect and differentiate the antibodies against the COVID-N and COVID-S protein. Since most COVID-19 vaccines target COVID-19 S protein, presence of antibodies to COVID-N protein indicates a natural infection regardless of whether there is antibody against the S protein. Presence of antibodies to the S protein only indicates that the sample is from an individual who has been vaccinated with a vaccine targeting the S protein and has not experienced a natural infection.

The test can be done with serum, plasma or whole blood. Whole blood from a fingerstick is provides an adequate volume of blood for the test. The test is suitable for use in CLIA-Waived Point-of-Care (“POC”) settings. The assay semi-quantitatively determines the amount of S and N protein in the serum, plasma or blood sample. The amount of the S and N proteins relative to one another differentiates antibodies developed in response to SARS-CoV-2 infection from those generated in response to inoculation with an S-protein based vaccine.

Test can be stored at room temperature (22-28° C.) and typically are provided in a kit, including a test cassette, sample buffer, and transfer pipettes. Kits can be shipped at ambient temperature, and are stable for at least seven days at 45° C.

Results in are colorimetric.

Sensitivity and specificity for S protein detection in naturally infected patients (>14 days): >/=95% and 98%, respectively.

Sensitivity and specificity for N protein detection in naturally infected patients (>14 days): >/=95% and 98%, respectively.

The presence of antibodies to COVID-N protein indicates a natural infection regardless of whether there is antibody against the S protein. Presence of antibodies to COVID-S protein only but not to COVID-19 N protein indicates vaccination with a vaccine targeting the S protein and no natural infection. Presence of antibodies to both N and S proteins indicates a natural infection and/or vaccination of a vaccine targeting the S protein. Since vaccination is known to the individual from whom the test sample is collected, the vaccination information can be used to further refine interpretation of test results. Interpretation of the test results are summarized in Table 1.

TABLE 1
Interpretation of Test Results

| | Antibody | Antibody | | |
|---|---|---|---|---|
| | to | to | | |
| | COVID-19 | COVID-19 | | |
| | Virus N | Virus S | Vaccination | |
| | Protein | Protein | Status | Interpretation |
| 1 | Negative | Negative | Vaccinated with a | No antibody indicative of |
| | | | vaccine targeting | natural infection or |
| | | | S protein | vaccination is detected |
| 2 | Negative | Positive | Vaccinated with a | No antibody indicative of |
| | | | vaccine targeting | natural infection is |
| | | | S protein | detected; antibody |
| | | | | indicative of vaccination is |
| | | | | detected |
| 3 | Positive | Negative | Vaccinated with a | Antibody indicative of |
| | | | vaccine targeting | natural infection is |
| | | | Sprotein | detected; no antibody |
| | | | | indicative of vaccination is |
| | | | | detected |
| 4 | Positive | Positive | Vaccinated with a | Antibodies indicative of |
| | | | vaccine targeting | natural infection and |
| | | | S protein | vaccination are detected. |
| 5 | Negative | Negative | No vaccination of | No antibody indicative of |
| | | | any type | natural infection is detected |
| 6 | Negative | Positive | No vaccination of | Antibody indicative of |
| | | | any type | natural infection is detected |
| 7 | Positive | Negative | No vaccination of | Antibody indicative of |
| | | | any type | natural infection is detected |
| 8 | Positive | Positive | No vaccination of | Antibody indicative of |
| | | | any type | natural infection is detected. |

### BIBLIOGRAPHYT

The following publications provide background information on various aspects of the inventions herein disclosed. Each and all of these publications is incorporated herein by reference, particularly in part pertinent to understanding aspects and particular embodiments of inventions herein disclosed.

(1) Singh et al. (2021): Microstructure, pathophysiology, and potential therapeutics of COVID-19: A comprehensive review; J Med Virol 93(1) 275-299; doi: 10.1002/jmv.26254; PMID: 32617987 PMCID: PMC7361355.

(2) Chau et al. (2020): COVID-19 Clinical Diagnostics and Testing Technology; Pharmacotherapy 40(8) 857-868; doi: 10.1002/phar.2439; PMID: 32643218 PMCID: PMC7361586.

(3) Abubakar et al. (2021): Coronavirus disease 2019 (COVID-19): An overview of the immunopathology, serological diagnosis and management; Scand J Immunol 93(4) e12998; doi: 10.1111/sji.12998; PMID: 33190302 PMCID: PMC7744910.

(4) Jiayue et al. (2020): Serological antibody testing in the COVID-19 pandemic: their molecular basis and applications; Biochem Soc Trans 48(6):2851-2863; doi:10.1042/BST20200744, PMID: 33170924.

(5) Yamayoshi et al. (2020): Comparison of Rapid Antigen Tests for COVID-19; Viruses 12(12) 1420; doi: 10.3390/v12121420. PMID: 33322035

(6) Machado et al. (2020): The Main Molecular and Serological Methods for Diagnosing COVID-19: An Overview Based on the Literature; Viruses 13(1):40; doi: 10.3390/v13010040. PMID: 33383888

(7) Xie et al. (2020): Characteristics of patients with coronavirus disease (COVID-19) confirmed using an IgM-IgG antibody test; J Med Virol 92(10):2004-2010; doi: 10.1002/jmv.25930, PMID: 32330303

(8) V'kovski et al. (2020): Coronavirus biology and replication: implications for SARS-CoV-2; Nat Rev Microbiol 19(3):155-170; doi: 10.1038/s41579-020-00468-6; PMID: 33116300 PMCID: PMC7592455.

(9) Boris G. Andryukov (2020): Six decades of lateral flow immunoassay: from determining metabolic markers to diagnosing COVID-19; AIMS Microbiol 6(3) 280-304; doi: 10.3934/microbiol.2020018, PMCID: PMC7595842, PMID: 33134745.

(10) Koczula et al. (2016): Lateral flow assays; Essays Biochem 60(1) 111-120; doi: 10.1042/EBC20150012, PMCID: PMC4986465, PMID: 2736504.

What is claimed is:

1. A test strip, comprising:
a first separate and distinct area that indicates the presence or absence in a sample of antibodies to a first SARS-CoV-2 protein;
a second separate and distinct area that indicates the presence or absence in a sample of antibodies to a second SARS-CoV-2 protein different from the first protein;
a third separate and distinct area that is a control indicating whether or not the test functioned properly.

2. A test strip according to claim 1, wherein
said first area has immobilized thereon at least one of the following proteins or an epitope-containing fragment thereof: a SARS-CoV-2 S protein, M protein, E protein or N protein;
said second area has immobilized thereon at least one of the following proteins or an epitope-containing fragment thereof: a SARS-CoV-2 S protein, M protein, E protein or N protein;
wherein said SARS-CoV-2 proteins or fragments immobilized on said first area are different from said SARS-CoV-2 proteins or fragments immobilized on said second area.

3. A test strip according to claim 1, further comprising:
a fourth separate and distinct area having immobilized thereon at least one non-SARS-CoV-2 Coronavirus protein or epitope-containing fragment thereof.

4. A test strip according to claim 2, further comprising:
a fourth separate and distinct area having immobilized thereon at least one non-SARS-CoV-2 Coronavirus protein or epitope-containing fragment thereof.

5. A test strip according to claim 1, further comprising:
a fifth separate and distinct area having therein in soluble form at least one non-SARS-CoV-2 Coronavirus protein or epitope containing fragment thereof;
wherein the sample contacts said fifth area before contacting said first, second and third areas on the strip.

6. A test strip according to claim 2, further comprising:
a fifth separate and distinct area having therein in soluble form at least one non-SARS-CoV-2 Coronavirus protein or epitope containing fragment thereof;
wherein the sample contacts said fifth area before contacting said first, second and third areas on the strip.

7. A test strip according to claim 3, wherein the non-COVID-19 virus Coronavirus is one or more of Coronavirus OCE43, 299E, HKD1 and NL63.

8. A test strip according to claim 4, wherein the non-COVID-19 virus Coronavirus is one or more of Coronavirus OCE43, 299E, HKD1 and NL63.

9. A test strip according to claim 5, wherein the non-COVID-19 virus Coronavirus is one or more of Coronavirus OCE43, 299E, HKD1 and NL63.

10. A test strip according to claim 6, wherein the non-COVID-19 virus Coronavirus is one or more of Coronavirus OCE43, 299E, HKD1 and NL63.

11. A test strip according to claim 1, further comprising:
a separate and distinct sample application area,
a separate and distinct conjugate contact area comprising labeled antibody binding reagents, wherein
sample applied to the sample application area contacts the conjugate contact area before contacting the first, second and third areas, whereby,
the labeled antibody binding reagents bind to antibodies in the sample, and the presence of absence of antibodies to SARS-CoV-2 is detected by the presence or absence of the detectable label binding to areas having immobilized thereon the SARS-CoV-2 proteins or fragments thereof.

12. A test strip according to claim 2, further comprising:
a separate and distinct sample application area,
a separate and distinct conjugate contact area comprising labeled antibody binding reagents, wherein
sample applied to the sample application area contacts the conjugate contact area before contacting the first, second and third areas, whereby,
the labeled antibody binding reagents bind to antibodies in the sample, and the presence of absence of antibodies to SARS-CoV-2 is detected by the presence or absence of the detectable label binding to areas having immobilized thereon the SARS-CoV-2 proteins or fragments thereof.

13. A test strip according to claim 11, wherein the label is colloidal gold.

14. A test strip according to claim 12, wherein the label is colloidal gold.

15. A test strip according to claim 1, wherein the SARS-CoV-2 protein immobilized in the first area is the S protein or an epitope-containing fragment thereof.

16. A test strip according to claim 2, wherein the SARS-CoV-2 protein immobilized in the first area is the S protein or an epitope-containing fragment thereof.

17. A test strip according to claim 15, wherein the SARS-CoV-2 protein immobilized in the second area is the E protein or an epitope-containing fragment thereof.

18. A test strip according to claim 16, wherein the SARS-CoV-2 protein immobilized in the second area is the E protein or an epitope-containing fragment thereof.

19. A test strip according to claim 15, wherein the SARS-CoV-2 protein immobilized in the second area is the M protein or an epitope-containing fragment thereof.

20. A test strip according to claim 16, wherein the SARS-CoV-2 protein immobilized in the second area is the M protein or an epitope-containing fragment thereof.

21. A test strip according to claim 15, wherein the SARS-CoV-2 protein immobilized in the second area is the N protein or an epitope-containing fragment thereof.

22. A test strip according to claim 16, wherein the SARS-CoV-2 protein immobilized in the second area is the N protein or an epitope-containing fragment thereof.

23. A test strip comprising:
a first separate and distinct area having thereon in soluble form one or more detectably labelled SARS-CoV-2 virus proteins or fragments thereof;
a second separate and distinct area having immobilized thereon an antibody binding agent that binds specifically to human IgG antibodies;
a third separate and distinct area having immobilized thereon an antibody binding agent that binds specifically to human IgM antibodies;
a fourth separate and distinct area the indicates the positive or negative outcome of a positive control and thereby whether the test functioned properly;
wherein a sample applied to the test contacts the first area before contacting the second and third areas,
whereby anti-SARS-CoV-2 IgG antibodies in the sample that bind to detectably labeled SARS-CoV-2 proteins in the first area are indicated by detectable label binding to the second area and
anti-SARS-CoV-2 IgM antibodies in the sample that bind to detectably labeled SARS-CoV-2 proteins in the first area are indicated by detectable label binding to the second area.

24. A test strip according to claim 23, wherein the first area further comprises proteins of a non-SARS-CoV-2 Coronavirus that are not detectably labeled.

25. A device for detecting antibodies to SARS- CoV-2 in a sample, comprising,
at least one test strip according to claim 1,
a port for applying a sample to a sample application area on the strip;
a source of buffer disposed upstream of the sample application;
and windows for detecting the presence or absence of detectable label binding to the SARS-CoV-2 and/or non-SARS-CoV-2 Coronavirus proteins and/or fragments immobilized in the respective separate and distinct areas thereon.

26. A device for detecting antibodies to SARS-CoV-2 in a sample, comprising,
at least one test strip according to claim 2,
a port for applying a sample to a sample application area on the strip;
a source of buffer disposed upstream of the sample application;
and windows for detecting the presence or absence of detectable label binding to the SARS-CoV-2 and/or non-SARS-CoV-2 Coronavirus proteins and/or fragments immobilized in the respective separate and distinct areas thereon.

27. A device for detecting anti-SARS-CoV-2 antibodies in a sample, comprising:
at least one test strip according to claim 21,
a port for applying a sample to a sample application area on the strip;
a source of buffer disposed upstream of the sample application;
and windows for detecting the presence or absence of detectable label binding to the anti-IgG and anti-IgM antibodies immobilized in the respective separate and distinct areas thereon.

28. A device for detecting anti-SARS-CoV-2 antibodies in a sample, comprising:
at least one test strip according to claim 24,
a port for applying a sample to a sample application area on the strip;
a source of buffer disposed upstream of the sample application;
and windows for detecting the presence or absence of detectable label binding to the anti-IgG and anti-IgM antibodies immobilized in the respective separate and distinct areas thereon.

29. A device according to claim 25, wherein the buffer pH is 9.5 to 11.5.

30. A device according to claim 26, wherein the buffer pH is 9.5 to 11.5.

31. A device according to claim 27, wherein the buffer pH is 9.5 to 11.5.

32. A device according to claim 28, wherein the buffer pH is 9.5 to 11.5.

33. A kit comprising
a device according to claim 25, and
a buffer solution of pH 9.5-11.5.

34. A kit comprising
a device according to claim 26, and
a buffer solution of pH 9.5-11.5.

35. A kit comprising
a device according to claim 27, and
a buffer solution of pH 9.5-11.5.

36. A kit comprising
a device according to claim 28, and
a buffer solution of pH 9.5-11.5

37. A method for determining the status of infection by SARS-CoV-2 virus in a subject vaccinated with a vaccine targeting the SARS-CoV-2 S protein or an epitope containing fragment of the S protein, comprising:
contacting an antibody-containing sample from a patient with a test strip, comprising:
a first separate and distinct area that indicates the presence or absence in a sample of antibodies to a first SARS-CoV-2 protein, wherein said first protein is targeted by a SARS-CoV-2 vaccine with which the subject has been inoculated;
a second separate and distinct area that indicates the presence or absence in a sample of antibodies to a second SARS-CoV-2 protein different from the first protein, wherein said second protein is not targeted by a SARS-CoV-2 vaccine with which the subject has been inoculated; and
a third separate and distinct area that is a control indicating whether or not the test functioned properly;
determining from said third area that the test functioned properly;
determining binding of antibodies in said sample to said first and said second area;
wherein binding to said first area relative to binding to said second area is indicative of the presence or absence of infection by SARS-CoV-2;
wherein binding to said second area relative to said first area exceeding a threshold value indicates the presence of infection by SARS-CoV-2, and below that value indicates an absence of infection by SARS-CoV-2.

38. A method according to claim37, wherein
said first area has immobilized thereon at least one of the following proteins or an epitope-containing fragment thereof: a SARS-CoV-2 S protein, M protein, E protein or N protein, wherein said one or more proteins is targeted by a SARS-CoV-2 vaccine to which the subject has been exposed;
said second area has immobilized thereon at least one of the following proteins or an epitope-containing fragment thereof: a SARS-CoV-2 S protein, M protein, E protein or N protein, wherein said one or more proteins is not targeted by a SARS-CoV-2 vaccine to which the subject has been exposed.

39. A method according to claim 38, wherein
said first area has immobilized thereon the SARS-CoV-2 S protein or an epitope-containing fragment of the S protein, and
said second area has immobilized thereon a SARS-CoV-2 N protein, or an epitope-containing fragment thereof.

40. A method according to claim 39, wherein binding of antibodies in the same to said proteins or epitope containing fragments is carried out a pH of 9.5 to 11.5.
